(19) United States
(12) Patent Application Publication
(10) Pub. No.: US 2025/0130228 A1
(43) Pub. Date: Apr. 24, 2025

(54) DEVICES AND METHODS FOR DETECTING ANALYTES

(71) Applicant: UNIQ BIOTECH LTD., Delta (CA)

(72) Inventors: Yunchuan ZHANG, Delta (CA); Yisha CHAI, Delta (CA)

(21) Appl. No.: 18/917,781

(22) Filed: Oct. 16, 2024

Related U.S. Application Data
(60) Provisional application No. 63/592,463, filed on Oct. 23, 2023.

(51) Int. Cl. G01N 33/543; G01N 21/78; G01N 33/76

(52) U.S. Cl. CPC G01N 33/54391; G01N 21/78; G01N 33/76; G01N 2333/59

(57) ABSTRACT

A device and method for detecting a plurality of analytes of interest in a sample are disclosed. The device has a first portion for detection of a first analyte of interest, and a second test portion for detection of a second analyte of interest. The first test portion has a test membrane with a chemical reaction side having a reaction mixture blotted thereon. The second test portion has a conjugate pad having detector particles bound thereon and an immunoreaction membrane having a test zone comprising a test antibody bound thereon, and a control zone comprising a control antibody bound thereon. The first test portion is in fluid-flow contact with the second test portion, arranged for transporting the sample away from the first test portion towards the second test portion.

### CROSS-REFERENCE TO RELATED APPLICATION

This application claims priority from U.S. application No. 63/592,463 filed Oct. 23, 2023 and entitled DEVICES AND METHODS FOR DETECTING ANALYTES which is hereby incorporated herein by reference for all purposes. For purposes of the United States of America, this application claims the benefit under 35 U.S.C. § 119 of U.S. application No. 63/592,463 filed Oct. 23, 2023 and entitled DEVICES AND METHODS FOR DETECTING ANALYTES which is hereby incorporated herein by reference for all purposes.

### FIELD OF THE INVENTION

The invention pertains to devices and methods for the detection of analytes in a sample of a subject, in particular, those that can semi-quantitatively detect such analytes.

### BACKGROUND

Devices and methods for detecting analytes of interest are known in the art. The present invention is directed to an improved device and method for detecting a plurality of analytes, in particular, one that combines in one device a chemical assay to detect one analyte of interest, and an immunoassay to detect another analyte of interest.

### SUMMARY

An aspect of the invention provides a device for detecting analytes of interest. The device comprises a first test portion and a second test portion arranged adjacent to the first test portion. The first test portion may be configured to detect a first analyte of interest, and the second test portion may be configured to detect a second analyte of interest. In some embodiments, the test first portion comprises a test membrane. The test membrane may comprise a chemical reaction side and an opposing filtration side. A reaction mixture may be blotted on the chemical reaction side of the test membrane. In some embodiments, the second test portion comprises a conjugate pad which comprises detector particles bound thereon, and an immunoreaction membrane in fluid-flow contact with the conjugate pad. In some embodiments, the immunoreaction membrane has a test antibody and a control antibody bound thereon. In some embodiments, the first test portion and the second test portion are in fluid-flow contact with one another. In some example embodiments, a sample pad is arranged in fluid-flow contact with the test membrane and the conjugate pad, for transporting the sample away from the first test portion towards the second test portion. In some embodiments, the sample pad is in contact with the chemical reaction side of the test membrane.

In some embodiments, a first analyte concentration indicator is arranged on a first surface of the device, adjacent to a chemical test signal zone. A test signal is produced at the chemical test signal zone if the first analyte of interest is present in the sample. The test signal may be a color indicator. In some embodiments, the intensity of the color of the test signal is directly proportional to the amount of the first analyte of interest present in the sample. The first analyte concentration indicator comprises a plurality of color indicators, each corresponding to a known concentration of the first analyte of interest.

In some embodiments, a second analyte concentration indicator is arranged on the first surface of the device, adjacent to an immunoreaction test signal zone. A test signal is produced at the immunoreaction test signal zone if the second analyte of interest is present in the sample. The test signal may be a color indicator. In some embodiments, the intensity of the color of the test signal is directly proportional to the amount of the second analyte of interest present in the sample. The second analyte concentration indicator comprises a plurality of color indicators, each corresponding to a known concentration of the second analyte of interest.

An aspect of the invention provides a method for detecting analytes of interest. In some embodiments, the method comprises loading a sample onto a sample loading zone and causing the sample to flow vertically onto a test membrane. The sample may be caused to chemically react with a reaction mixture blotted on the test membrane to produce a test signal at a chemical test signal zone. The test signal at the chemical test signal zone may be indicative of the presence of a first analyte of interest. The sample may then be caused to flow laterally from the test membrane onto a conjugate pad comprising detection particles bound thereon. The second analyte of interest, if present in the sample, may be caused to bind to the detection particles at the conjugate pad, thereby forming a complex. The detection particles and/or complex if formed may be released from the conjugate pad. The sample, the detection particles and/or complex if formed may be caused to flow laterally to an immunoreaction membrane comprising a test zone and a control zone. The test zone comprises one or more test antibodies and the control zone comprises one or more control antibodies. In some embodiments, the complex if formed may be caused to bind to the one or more test antibodies to produce a test signal at the test zone. The test signal is indicative of the presence of the second analyte of interest. In some embodiments, at least the detection particles are caused to bind to the one or more control antibodies at the control zone to produce a test signal.

In some embodiments, the method further comprises comparing a color intensity of the test signal produced at the chemical test signal zone with each of a plurality of color indicators that form a first analyte concentration indicator to semi-quantify a concentration of the first analyte of interest in the sample. Each of the plurality of color indicators may correspond to a known concentration of the first analyte of interest.

In some embodiments, the method further comprises comparing a color intensity of the test signal produced at the test zone of the immunoreaction membrane with each of a plurality of color indicators that form a second analyte concentration indicator to semi-quantify a concentration of the second analyte of interest in the sample. Each of the plurality of color indicators may correspond to a known concentration of the second analyte of interest.

Further aspects of the invention and features of specific embodiments of the invention are described below.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a top plan view of a device for detecting analytes according to an example embodiment of the invention.

FIG. 2 is a bottom plan view of the FIG. 1 device.

FIG. 3 is a cross-sectional view of the FIG. 1 device.

FIG. 4 is a flow chart illustrating a method for detecting analytes according to an example embodiment of the invention.

### DETAILED DESCRIPTION

### Example Embodiments of the Device

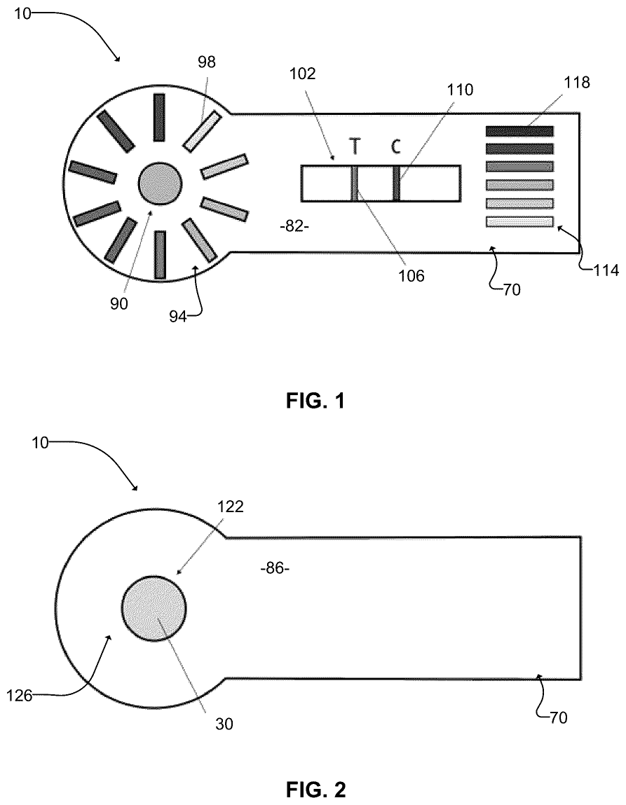

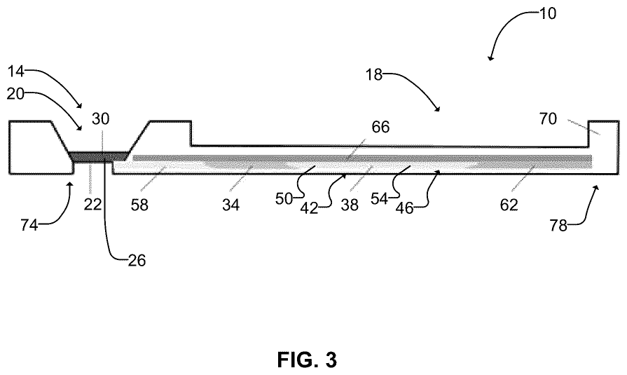

Referring to FIGS. 1-3, in one embodiment, the invention is a device 10 for detecting analytes of interest in a sample. The device 10 comprises a first test portion 14, and a second test portion 18 arranged adjacent to the first test portion 14. In some embodiments, the first and second test portions 14, 18 are configured to detect different analytes of interest. The first test portion 14 may be configured to detect a first analyte of interest. The second test portion 18 may be configured to detect a second analyte of interest. In some embodiments, the first test portion 14 is a vertical flow assay, and the second test portion 18 is a lateral flow assay. The device 10 combines a vertical flow assay with a lateral flow assay to detect a plurality of analytes in a sample.

In some embodiments, the first test portion 14 comprises a test membrane 20. The test membrane 20 may comprise a chemical reaction side 30. A reaction mixture 26 may be blotted on the chemical reaction side 30. The chemical reaction side 30 of the test membrane 20 may receive a fluid sample thereon. The fluid sample may undergo a chemical reaction with the one or more substances contained in the reaction mixture 26 on the chemical reaction side 30. In some embodiments, the test membrane 20 comprises a filtration side 22 opposite to the chemical reaction side 30. In some embodiments in which the sample comprises whole blood, the filtration side 22 of the test membrane 20 is adapted to separate, from the whole blood sample, serum from whole blood cells. The separated serum may then flow vertically from the filtration side 22 onto the chemical reaction side 30 to undergo one or more chemical reactions, and display a test signal thereon.

In some embodiments, the second test portion 18 comprises a conjugate pad 34 and an immunoreaction membrane 38 arranged in fluid flow contact with the conjugate pad 34. The conjugate pad 34 may be arranged laterally juxtaposed to the immunoreaction membrane 38, with at least some portions of the pad 34 and the membrane 38 overlapping one another. In some embodiments, the conjugate pad 34 comprises detector particles bound thereon. If the sample comprises the target analyte (i.e., the analyte which the detector particles is designed to recognize and bind thereto), the detector particles may bind to the target analyte, thereby forming a complex. In some example embodiments, the detector particles comprise an antibody conjugate. The antibody conjugate may comprise a tag or label bound to an antibody designed to specifically bind to the analyte of interest. The tag or label may comprise a nanoparticle. In one non-limiting example, the nanoparticle comprises gold nanoparticles.

The immunoreaction membrane 38 may comprise a test zone 42 and a control zone 46 arranged adjacent to the test zone 42. The test zone 42 comprises one or more test antibodies 50 immobilized thereon. The control zone 46 comprises one or more control antibodies 54 immobilized thereon. The one or more test antibodies 50 are designed to bind to the respective analytes of interest. In some embodiments, the test zone 42 is arranged more proximate to the first test portion 14 than the control zone 46 to the first test portion 14. In such embodiments, the sample which is caused to flow from the first test portion 14 to the second test portion 18, is caused to flow through the test zone 42 before flowing to the control zone 46.

In some embodiments, a sample pad 58 is arranged in fluid-flow contact with the test membrane 20 of the first test portion 14, and the conjugate pad 34 of the second test portion 18. In some embodiments, the sample pad 58 may be arranged vertically juxtaposed to the test membrane 20. In some embodiments, the sample pad 58 is arranged laterally juxtaposed to the conjugate pad 34. In some embodiments, the sample pad 58 is arranged to overlap at least a portion of each of the test membrane 20 and the conjugate pad 34. In some embodiments, the sample pad 58 contacts the chemical reaction side 30 of the test membrane 20. The sample pad 58 is arranged to transport the sample away from the first test portion 14 to the second test portion 18.

In some embodiments, the second test portion 18 comprises an absorbent pad 62 arranged in fluid-flow contact with the immunoreaction membrane 38 at a side opposite to the conjugate pad 34. The absorbent pad 62 is arranged to receive or collect the sample after the sample has been caused to be transported through the immunoreaction membrane 38.

In some embodiments, a holding pad 66 is arranged vertically juxtaposed to the sample pad 58 and/or the layers that form the second test portion 18. The holding pad 66 may be arranged to provide structural support to one or more such layers. In some embodiments, the holding pad 66 is arranged to overlap at least a portion of one or more of the sample pad 58, the conjugate pad 34, the immunoreaction membrane 38, and the absorbent pad 62. In the illustrated embodiments, the holding pad 66 is arranged to overlap at least a portion of the sample pad 58 along a longitudinal axis thereof. In some embodiments, the holding pad 66 is arranged to overlap substantially all of the conjugate pad 34, the immunoreaction membrane 38, and/or the absorbent pad 62.

A housing 70 may be provided, dimensioned to fit the first and second test portions 14, 18 and the sample pad 58 therein. In some embodiments, the first and second test portions 14, 18 and the sample pad 58 are aligned along a longitudinal axis of the housing 70. In some embodiments, the test membrane 20 is arranged at a first longitudinal zone 74 of the housing 70 and the absorbent pad 62 is arranged at a second opposing longitudinal zone 78 thereof.

As best illustrated in FIGS. 1 and 2, the device 10 has a first surface 82 and an opposing second surface 86. In some embodiments, a chemical test signal zone 90 is exposed on the first surface 82. The chemical test signal zone 90 is adapted to indicate a signal in response to the one or more chemical reactions that occurred at the chemical reaction side 30 of the test membrane 20. In some embodiments, the signal is in the form of a color. In some embodiments, the intensity of the color signal is directly proportional to the concentration of the first analyte of interest in the sample. In some embodiments, the chemical test signal zone 90 is at the chemical reaction side 30 of the test membrane 20.

In some embodiments, a first analyte concentration indicator 94 is provided on the first surface 82 of the device 10. The first analyte concentration indicator 94 may for example be imprinted on a surface of the housing 70. In some embodiments, the first analyte concentration indicator 94 comprises a plurality of color indicators 98. Each of the plurality of color indicators 98 may correspond to a known concentration of the first analyte of interest. In some embodiments, the first analyte concentration indicator 94 is arranged adjacent to the chemical test signal zone 90. In the illustrated embodiments, the plurality of color indicators 98 are arranged radially spaced-apart with respect to the chemical test signal zone 90. The first analyte concentration indicator 94 facilitates semi-quantitation of the first analyte of interest, by allowing a user to compare the intensity of the color signal shown on the chemical test signal zone 90 (upon causing the sample to flow through the chemical reaction membrane 22 so as to allow the sample to react with the reaction mixtures blotted on the chemical reaction membrane) with the intensities of the colors at each of the color indicators 98.

In some embodiments, an immunoreaction test signal zone 94 is exposed on the first surface 82 of the device 10. The immunoreaction test signal zone 102 may be positioned adjacent to the chemical test signal zone 90. The immunoreaction test signal zone 102 may comprise a test signal 106 and a control signal 110 adjacent to the test signal 106. The test signal 106 provides an indication of the presence of the second analyte of interest in the sample (e.g., by detecting the binding of the second analyte of interest to the specific antibody immobilized on the test zone 42). The control signal 110 provides an indication that the assay was valid (e.g., by detecting the binding of the detection particles such as conjugated antibodies to the non-specific antibody immobilized on the control zone 46). In some embodiments, the test signal 106 is in the form a color. In some embodiments, the intensity of the color signal is directly proportional to the concentration of the second analyte of interest in the sample.

In some embodiments, a second analyte concentration indicator 114 is provided on the first surface 82 of the device 10. The second analyte concentration indicator 114 may for example be imprinted on a surface of the housing 70. In some embodiments, the second analyte concentration indicator 114 comprises a plurality of color indicators 118. Each of the plurality of color indicators 118 may correspond to a known concentration of the second analyte of interest. In some embodiments, second analyte concentration indicator 114 is arranged adjacent to the immunoreaction test signal zone 102. In the illustrated embodiments, the plurality of color indicators 118 are arranged spaced-apart along a lateral axis of the device 10. The second analyte concentration indicator 114 facilitates semi-quantitation of the second analyte of interest, by allowing a user to compare the intensity of the color signal shown on the immunoreaction test signal zone 102 (upon causing the sample to flow through the conjugate pad 34 and the immunoreaction membrane 38 to allow for binding of the second analyte of interest and/or detection particles to the antibodies immobilized on the pad 34 and the membrane 38) with the intensities of the colors at each of the color indicators 118.

In some embodiments, a sample loading zone 122 is exposed on the second surface 86 of the device 10. In some embodiments, the sample loading zone 122 is at the chemical reaction side 30 and/or the filtration side 22 of the test membrane 20.

In some embodiments, one or more openings 126 may be defined on the housing 70 to allow one or more of the sample loading zone 122, the chemical test signal zone 90 and the immunoreaction test signal zone 102 to be exposed on the respective first and second surfaces 82, 86 of the device 10.

### Example Embodiments of the Method

Aspects of the invention pertains to a method of detecting analytes of interest. In some embodiments, the method of the invention involves combining one or more chemical reactions and one or more immunoreactions to detect a plurality of analytes of interest. In some embodiments, the method of the invention involves causing a sample to flow vertically in the detection of one analyte of interest, and causing the same sample to flow laterally in the detection of another analyte of interest. In some example embodiments, the method comprises detecting a first analyte of interest by one or more chemical reactions, and detecting a second analyte of interest by one or more immunoreactions.

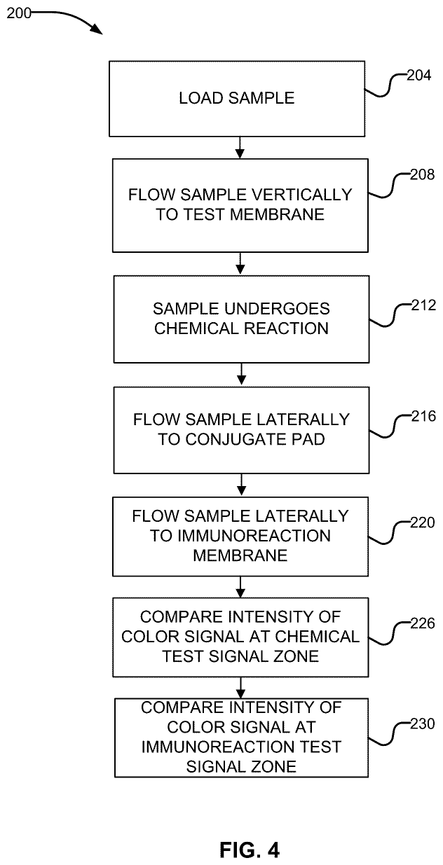

Referring to the flow chart in FIG. 4, an example method 200 comprises loading a sample onto a sample loading zone (block 204). The sample may then be caused to flow vertically, by gravity, onto a test membrane (block 208). The sample undergoes one or more chemical reactions, for example at the chemical reaction side of the first membrane, with the reaction mixture blotted on the test membrane (block 212). The reaction mixture comprises one or more compounds and/or substances suitable for the detection of a first analyte of interest. The presence of a test signal at the chemical test signal zone indicates the presence of the first analyte of interest in the sample. In some embodiments, the test signal comprises a color indicator. In such embodiments, the intensity of the color indicator may be directly proportional to the amount of the first analyte of interest present in the sample. In block 216, the sample may then be caused to flow laterally from the test membrane to a conjugate pad. The conjugate pad may comprise detection particles immobilized thereon, designed to bind to a second analyte of interest. If the second analyte of interest is present in the sample, the second analyte may bind to the detection particles, thereby forming a complex. The sample, the complex (if formed), and/or the detection particles released from the conjugate pad may then be caused to flow laterally to the immunoreaction membrane, through the test zone and the control zone (block 220). The immunoreaction membrane comprises at least one test antibody immobilized on the test zone and at least one control antibody immobilized on the control zone of the membrane. If the complex is formed (i.e., the second analyte of interest is present in the sample), the complex binds to the at least one test antibody. The presence of a test signal at the test zone indicates the presence of the second analyte of interest in the sample. In some embodiments, the test signal comprises a color indicator. In such embodiments, the intensity of the color indicator is directly proportional to the amount of the second analyte of interest present in the sample. The presence of a control signal at the control zone indicates that the released detection particles have bound to the at least one control antibody, thereby suggesting that the immunoassay is valid. The absence of a control signal indicates that the released detection particles have not bound to the at least one control antibody, thereby suggesting that the immunoassay is invalid.

In some embodiments, the method comprises preparing a sample prior to causing the sample to contact the test membrane. In some example embodiments, the preparing of the sample comprises separating red blood cells from the serum contained in a whole blood sample by loading the sample onto a filtration side of the test membrane. In such embodiments, the red blood cells remain on the filtration side while the serum flows through the filtration side towards the chemical reaction side of the test membrane. The chemical test signal may be present on the chemical reaction side.

In some embodiments, the method comprises comparing the intensity of the color signal formed at the chemical test signal zone with the plurality of the color indicators which form a first analyte concentration indicator, thereby semi-quantifying the first analyte of interest contained in the sample (block 226). Each of the plurality of color indicators correspond to a known concentration of the first analyte of interest.

In some embodiments, the method comprises comparing the intensity of the test signal formed at the immunoreaction test signal zone with the plurality of the color indicators which form a second analyte concentration indicator, thereby semi-quantifying the second analyte of interest contained in the sample (block 230). Each of the plurality of color indicators correspond to a known concentration of the second analyte of interest.

The following section discusses one non-limiting example application of the devices 10 and methods 200; however, it would be apparent to a skilled person that the devices 10 and the methods 200 of the present invention may be used and/or applied to detect any suitable plurality of analytes of interest, including but not limited to glucose and hCG levels, infectious disease detection (e.g., HIV/HBV/HCV antibody test and antigen test), etc., from any suitable sample, including but not limited to blood (e.g., whole blood and serum), urine, etc.

### Non-Limiting Example Application

One non-limiting example application of the device 10 is in the detection of glucose and human chorionic gonadotropin (hCG or b-hCG) in a sample. The sample may be a blood sample from a subject, such as a human subject. The device 10 advantageously combines the detection of glucose and hCG in one device with only one sample. The device 10 also advantageously allows for the semi-quantification of the concentrations of glucose and hCG contained in the sample using one sample.

The ability to detect and semi-quantify glucose and hCG levels quickly from one sample is particularly beneficial to pregnant women.

Gestational diabetes mellitus (GDM) affects between 3% and 20% of pregnant women. It presents with a rise in blood glucose (sugar) levels toward the end of the 2nd and 3rd trimester of pregnancy. In 90% of cases, it disappears after the birth, but the mother is at greater risk of developing type 2 diabetes in the future. The Canadian Diabetes Association 2018 Clinical Practice Guidelines for the Prevention and Treatment of Diabetes in Canada recommends diabetes screening for all pregnant women, between the 24th and 28th week of pregnancy. Women with a higher risk of developing gestational diabetes should be tested earlier and more frequently. The risk factors include for example, being 35 years of age or older; form a high-risk group; using corticosteroid medication; having obesity, prediabetes, gestational diabetes in a previous pregnancy, etc. For example, self-monitoring of blood glucose 4 to 7 times per day is also recommended for pregnant women with type 2 diabetes (i.e. fasting, preprandial and 1 or 2 hours postprandially) to achieve good glycemic control.

Human chorionic gonadotropin (hCG or b-hCG) is a hormone that the placenta produces when a woman is pregnant. hCG appears in the blood and urine of pregnant women as early as 10 days after conception. Quantitative hCG measurement helps determine the exact age of the fetus. It can also assist in the diagnosis of abnormal pregnancies, such as ectopic pregnancies, molar pregnancies, and possible miscarriages. Beta-hCG can be monitored periodically during the first trimester and second trimesters in high-risk pregnancies when there is a history or risk of miscarriage. A beta-hCG test is also used when there are concerns about pregnancy complications, including miscarriage. In these situations, repeat tests may be performed every two to three days to evaluate how quickly hCG levels are rising. Early on in pregnancy, the rate of increase is more telling than the actual quantity of the hormone in the blood. Slow-to-rise hCG levels may indicate a high risk for miscarriage.

In some embodiments of the device 10, the first test portion 14 is adapted to detect the presence of glucose in the sample. In such embodiments, the reaction mixture 26 blotted on the chemical reaction side 30 of the test membrane 20 comprises one or more enzymes. In some embodiments, the one or more enzymes comprise a glucose oxidase. In some embodiments, the one or more enzymes comprise one or more peroxidases. The one or more peroxidases may for example comprise horseradish peroxidase (HRP). In some embodiments, the reaction mixture 26 comprises a chromogenic substrate. The chromogenic substrate may for example be 3,3′,5,5′-tetramethylbenzidine (TMB). In embodiments in which the reaction mixture 26 comprises glucose oxidase, TMB, and a peroxidase, it is believed that the following two chemical reactions occur on the chemical reaction side 30 of the test membrane 20 when the sample containing glucose is provided thereon:

Glucose+O₂+H₂O→Gluconic Acid+H₂O₂ Eq. 1

H₂O₂+TMB→Blue diimine-diamine complex+H₂O Eq. 2

Referring to Eq. 1, glucose is oxidized to gluconic acid and hydrogen peroxide (H₂O₂) in the presence of glucose oxidase. Referring to Eq. 2, the produced hydrogen peroxide then react with TMB, which can act as a hydrogen donor for the reduction of hydrogen peroxide to water by peroxidase enzymes (e.g., horseradish peroxidase (HRP)). The oxidized product is a diimine-diamine complex, which causes the solution to turn into a blue color. The intensity of the blue color formed may be directly proportional to the amount of glucose present in the sample.

In some embodiments, the first analyte concentration indicator 94 comprises a plurality of color indicators 98, where each color indicator 98 corresponds to a specific glucose concentration. In some example embodiments, the first analyte concentration indicator 94 comprises a plurality of color indicators 98, each of the color indicators 98 corresponding to a glucose concentration in the range of from about 0 mmol/L to about 25 mmol/L. In some example embodiments, the plurality of color indicators 98 comprises ten color indicators 98, and each of the color indicators 98 corresponds to a glucose concentration of one of about 0.1 mmol/L, 2.5 mmol/L, about 4.5 mmol/L, about 6.5 mmol/L, about 8.0 mmol/L, about 10.0 mmol/L, about 13.0 mmol/L, about 17.0 mmol/L, about 22.0 mmol/L, and about 25 mmol/L.

In some embodiments of the device 10, the second test portion 18 is adapted to detect the presence of beta-hCG in the sample. In such embodiments, the conjugate pad 34 comprises a conjugated antibody, such as an anti-beta hCG antibody conjugated to a gold nanoparticle (i.e., anti-beta hCG conjugated gold nanoparticle) immobilized thereon. The anti-beta hCG conjugated gold nanoparticles are designed to bind to hCG hormones present in the sample. If hCG hormones are present in the sample, the hCG hormones will bind to the anti-beta hCG conjugated gold nanoparticles, forming a beta hCG-anti-beta hCG antibody complex. In some embodiments, the test antibody 50 immobilized on the immunoreaction membrane 38 comprises an anti-beta hCG antibody. The anti-beta hCG antibody is designed to bind to the beta hCG-anti-beta hCG antibody complex (if formed) that is released from the conjugate pad 34. The control antibody 54 may be any suitable isotype control antibodies that lack specificity to the target analyte. In some example embodiments, the control antibody 54 comprises a mouse anti-human IgG antibody.

In some embodiments, the second analyte concentration indicator 114 comprises a plurality of color indicators 118, where each color indicator 118 corresponds to a specific hCG concentration. In some example embodiments, the second analyte concentration indicator 114 comprises a plurality of color indicators 118, each of the color indicators 118 corresponding to a hCG concentration in the range of from 0 IU/L to about 210,000 IU/L. In some embodiments, the plurality of color indicators 118 comprises six color indicators 118, and each of the color indicators 118 corresponds to a glucose concentration of one of 750 IU/L, 7,000 IU/L, 32,000 IU/L, 60,000 IU/L, 160,000 IU/L, and 210,000 IU/L.

In an example use embodiment, a whole blood sample is drawn from the subject, and loaded onto the sample loading zone 122. The whole blood sample is caused to flow through the filtration side 22 of the test membrane 20 towards the opposite chemical reaction side 30 of the test membrane 20. The filtration side 22 may be adapted to separate the red blood cells from the serum, and the separated serum may be caused to be flow to the chemical reaction side 30. The flow of the separated serum to the chemical reaction side 30 may be a vertical flow by gravity. The serum may then be caused to react with the reaction mixture 26 blotted on the chemical reaction side 30. In some embodiments, the glucose in the serum is oxidized by glucose oxidase, producing hydrogen peroxide. The hydrogen peroxide may then react with TMB by peroxide which produces a blue color at the chemical test signal zone 90. The amount of glucose present in the sample may be determined by comparing the intensity of the blue color to the color indicators 98 of the first analyte concentration indicator 94. The serum may then be caused to flow through the sample pad 58 to the conjugate pad 34. If hCG hormones are present in the sample, the hCG hormones will bind to the anti-beta hCG conjugated gold nanoparticles immobilized on the conjugate pad 34, thereby forming a beta hCG-anti-beta hCG antibody complex. The beta hCG-anti-beta hCG antibody complex may then be released from the conjugate pad 58 and be caused to flow to the immunoreaction membrane 38. If the complex is present (i.e., if the sample comprises hCG), the complex will bind to the anti-hCG antibody immobilized on the test zone 42, resulting in a colored test signal 106. The amount of hCG present in the sample may be determined by comparing the intensity of the color shown at the test signal 106 to the color indicators 118 of the second analyte concentration indicator 114. The anti-beta hCG conjugated gold nanoparticles will bind to the control antibody 54 (e.g., an isotype control antibody) immobilized on the control zone 46, resulting in a colored control signal 110. The absence of a control signal 110 at the control zone 46 is an indication that the assay is invalid.

Throughout the foregoing description and the drawings, in which corresponding and like parts are identified by the same reference characters, specific details have been set forth in order to provide a more thorough understanding to persons skilled in the art. However, well known elements may not have been shown or described in detail or at all to avoid unnecessarily obscuring the disclosure.

As will be apparent to those skilled in the art in the light of the foregoing disclosure, many alterations and modifications are possible in the practice of this invention without departing from the scope thereof. Accordingly, the description and drawings are to be regarded in an illustrative, rather than a restrictive, sense.

1. A device (10) for detecting analytes of interest in a sample, comprising:
a first test portion (14) for detection of a first analyte of interest, the first test portion comprising a test membrane (20) having a chemical reaction side (30), comprising a reaction mixture blotted thereon;
a second test portion (18) for detection for a second analyte of interest, the second test portion comprising:
a conjugate pad (34) comprising detector particles bound thereon; and
an immunoreaction membrane (38) in fluid-flow contact with the conjugate pad, the immunoreaction membrane comprising a test zone (42) comprising a test antibody (50) bound thereon, and a control zone (46) comprising a control antibody (54) bound thereon,
wherein the first test portion is in fluid-flow contact with the second test portion, arranged for transporting the sample away from the first test portion towards the second test portion.

2. The device as defined in claim 1, wherein the first and second test portions are in fluid-flow contact by arranging a sample pad (58) in fluid-flow contact with the test membrane and the conjugate pad.

3. The device as defined in claim 2, wherein the sample pad is arranged in contact with the chemical reaction side of the test membrane.

4. The device as defined in claim 1, wherein the second test portion further comprises an absorbent pad (62) in fluid-flow contact with the immunoreaction membrane, arranged to receive the sample flowing from the immunoreaction membrane.

5. The device as defined in claim 1, wherein the test membrane comprises a filtration side (22) opposite to the chemical reaction side.

6. The device as defined in claim 1, wherein the second test portion further comprises a holding pad (66) arranged to overlap at least a portion of one or more of the sample pad, the conjugate pad, the immunoreaction membrane and the absorbent pad.

7. The device as defined in claim 1, comprising a housing (70) dimensioned to fit one or more of the first test portion, the second test portion and the sample pad therein.

8. The device as defined in claim 1, wherein the first test portion, the second test portion and the sample pad are aligned along a longitudinal axis of the housing.

9. The device as defined in claim 6, wherein the holding pad is vertically juxtaposed to one or more of the sample pad, conjugate pad, the immunoreaction membrane and the absorbent pad.

10. The device as defined in claim 7, wherein the device comprises a first surface (82) and an opposing second surface (86), and wherein a chemical test signal zone (90) is exposed on the first surface of the device, and/or a sample loading zone (122) is exposed on the second surface of the device.

11. The device according to claim 10, wherein an immunoreaction test signal zone is exposed on the first surface of the device, laterally adjacent to the chemical test signal zone.

12. The device according to claim 10, further comprising a first analyte concentration indicator (94) on the first surface of the device, adjacent to the chemical test signal zone, and a second analyte concentration indicator (114) on the first surface of the device, adjacent to the immunoreaction test signal zone.

13. The device according to claim 12, wherein the first and second analyte concentration indicators comprises a plurality of color indicators (98, 118), each of the plurality of color indicators corresponding to a known concentration of the respective first and second analytes.

14. The device according to claim 13, wherein the plurality of color indicators of the first analyte concentration indicator are positioned radially with respect to the chemical test signal zone, each of the plurality of color indicators being arranged spaced-apart from each other.

15. The device according to claim 1, wherein the first analyte of interest is blood glucose, and wherein the reaction mixture blotted on the chemical reaction side of the test membrane comprises one or more enzymes.

16. The device according to claim 1, wherein the reaction mixture blotted on the chemical reaction membrane comprises a chromogenic substrate.

17. The device according claim 1, wherein the second analyte of interest is human chorionic gonadotropin (hCG or b-hCG), and wherein the detector particles bound on the conjugate pad comprises a conjugated antibody.

18. The device according to claim 17, wherein the conjugated antibody comprises an anti-beta hCG antibody conjugated to a gold nanoparticle.

19. The device according to claim 1, wherein the test antibody bound on the immunoreaction membrane comprises an anti-beta hCG antibody.

20. The device according to claim 13, wherein an intensity of the color shown in the color indicator of the second analyte concentration indicator increases with an increase in hCG concentration.
